(19) United States
(12) Patent Application Publication
(10) Pub. No.: US 2006/0104928 A1
(43) Pub. Date: May 18, 2006

(54) Thermal hair straightening and conditioning composition

(76) Inventor: Lucimar Furtado, Coral Springs, FL (US)

Correspondence Address: Mark D. Bowen;Stearns, Weaver, Miller, Weissler,, Alhadeff & Sitterson PA., 200 East Broward Boulevard, Suite 1900, Fort Lauderdale, FL, 33301, US

(21) Appl. No.: 10/987,895

(22) Filed: Nov. 12, 2004

(51) Int. Cl. A61K 8/64

(52) U.S. Cl. 424/70.2

(57) ABSTRACT

A thermal hair straightening and conditioning composition and method for rendering hair more manageable, minimizing static charge, tangling, and scattering of the hair is disclosed. A gelatinous aqueous thermal hair straightening and conditioning composition is provided that imparts a firm and silky look and feel to hair and inhibits wind and motion induced hair scattering during use. The composition comprises amino acids of human hair keratin, bio-extenders, oligo elements of marine algae, vitamin-E, silicones with orange extract placenta, formaldehyde, water, formic acid, isotiazolomas, citric acid, and fragrance. The hair straightener and conditioner composition of the invention preferably is used during hot iron and after hot iron straightening of curly hair, to impart a smooth and silky feel to the hair.

### CROSS REFERENCE TO RELATED APPLICATIONS

N/A

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyrights rights whatsoever.

### BACKGROUND OF THE INVENTION

1. Field of the Invention

The present invention relates to the straightening and conditioning of hair, and more particularly to a thermal hair straightening and conditioning composition for use in hot iron straightening methods.

2. Description of Related Art

Consumers often apply a thermal hair straightening and conditioning composition to hair during make-overs and hair styling. Conventional hair straightening and conditioning compositions generally contain man-made chemicals that often leave the hair undesirably harsh, dull, and dry. If left untreated, hair often becomes tangled when wet and/or dry such that combing and brushing of the hair is difficult. Furthermore, hair frequently exhibits poor electrostatic properties such that it is susceptible to “scattering”, especially in low humidity environments.

It is also common to employ the use of a hot iron comb in an effort to tame and straighten hair. However, hair subjected to the abuse of a hot iron comb sometimes becomes damaged due to the intense heat and restructuring of the hair stem by the heat.

Thus, to address the straightening and conditioning properties of the hair, both in the wet and dry states, there exists a need for a thermal hair straightening and conditioning composition that is applied before and after thermal straightening to render the hair more manageable, to minimize static charge, tangling, and scattering of the hair. There further exists a need for such a composition that is effective in changing hair texture when applied after washing of the hair. Such a composition should preferably transform dry damaged and frizzy hair by hydration such that the hair becomes silky and straight after application.

### BRIEF SUMMARY OF THE INVENTION

The present invention overcomes the limitations and disadvantages in the art of hair care by providing a thermal hair straightening and conditioning composition that is applied before and after thermal straightening to render the hair more manageable, to minimize static charge, tangling, and scattering of the hair. The present invention further provides a composition that is effective in changing hair texture when applied after washing of the hair. A composition according to the present invention transforms dry damaged and frizzy hair by hydration such that the hair becomes silky and straight after application.

In a preferred embodiment, the present invention provides a gelatinous aqueous thermal hair straightening and conditioning composition that preferably imparts a firm and silky look and feel to hair and inhibits wind and motion induced hair scattering during use. The composition comprises amino acids of human hair keratin, bio-extenders, oligo elements of marine algae, vitamin-E, silicones with orange extract placenta, formaldehyde, water, formic acid, isotiazolomas, citric acid, and fragrance. The hair straightener and conditioner composition of the invention preferably is used during hot iron and after hot iron straightening of curly hair, and imparts a smooth and silky feel to hair.

Accordingly, it is an object of the present invention to provide an improved thermal hair straightening and conditioning composition.

Still another object of the present invention is to provide a hair straightening and conditioning composition for use during in hot iron straightening of curly hair.

In accordance with these and other objects, which will become apparent hereinafter, the instant invention will now be described with particular reference to the accompanying drawings.

### BRIEF DESCRIPTION OF THE SEVERAL VIEWS OF THE DRAWINGS

FIG. 1 discloses an example of a composition according to a preferred embodiment of the present invention; and

FIG. 2 discloses a second example of a composition according to a preferred embodiment of the present invention.

### DETAILED DESCRIPTION OF THE INVENTION

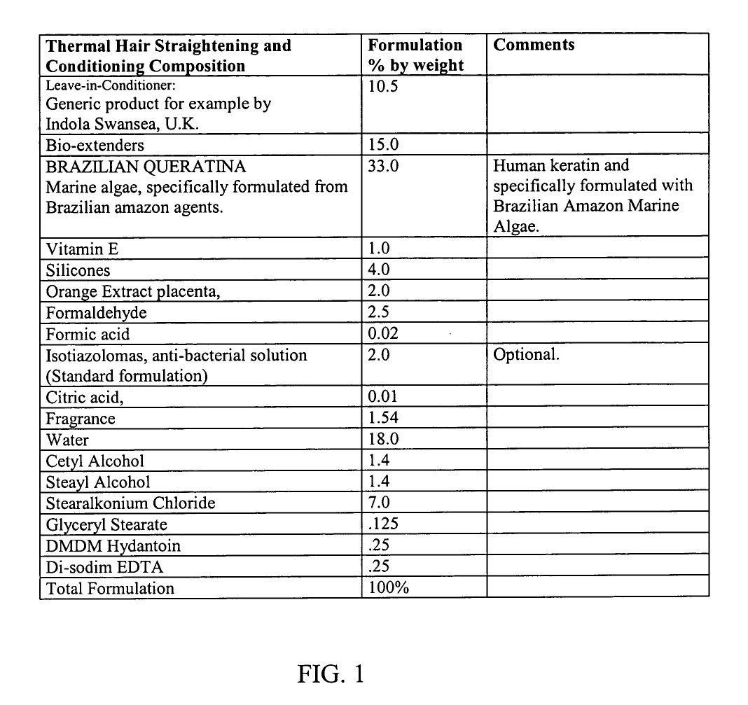

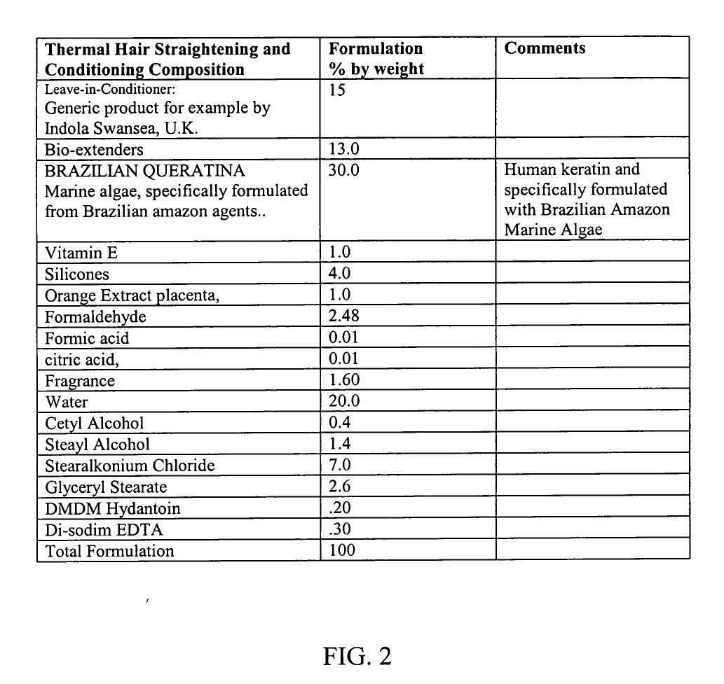

With reference now to the drawings, FIGS. 1 and 2 disclose chemical compositions for the present invention. Compositions are provided for use in the art of hair care wherein a thermal hair straightening and conditioning composition is applied before and after thermal straightening to render the hair more manageable, to minimize static charge, tangling, and scattering of the hair. The disclosed compositions are also effective in changing hair texture when applied after washing of the hair. It has further been found that a composition according to the present invention transforms dry damaged and frizzy hair by hydration such that the hair becomes silky and straight after application.

In a preferred embodiment, the present invention provides a gelatinous aqueous thermal hair straightening and conditioning composition that preferably imparts a firm and silky look and feel to hair and inhibits wind and motion induced hair scattering during use. The composition comprises amino acids of human hair keratin, bio-extenders, oligo elements of marine algae, vitamin-E, silicones with orange extract placenta, formaldehyde, water, formic acid, isotiazolomas, citric acid, and fragrance. The hair straightener and conditioner composition of the invention preferably is used during hot iron and after hot iron straightening of curly hair, and imparts a smooth and silky feel to hair.

The following example illustrates the improved properties of heat straightening and conditioning compositions of the above composition during and after application. A random test group of 8 women each with a different type of hair, ranging from silky straight hair to very curly and damaged hair was used at a well known hair salon. A variety of different styles of hair was used for the study. The straightener and conditioner as per the above composition was applied before and after the hot iron comb straightening method on curly hair.

The hot iron comb with a wide-tooth comb as commonly used in the hair care business was used to evaluate the effects. The comb was pre-heated by an electric heater and then the above-referenced composition was applied to lightly wetted hair. The hair was straightened and then the solution was reapplied to the finished hair with a leave-in conditioner. The conditioner is a generic off-the shelf conditioner.

The dried straightened hair was evaluated with respect to the presence of electrostatic charge scatter at low humidity of about 38% Relative Humidity, 72 degree F. and at high humidity of about 78% Relative humidity, 72 degrees F. The straightener was tested using a blow dryer to evaluate the silkiness. After each month of application, the hair was inspected after repeated washes and found to be still silky and straight. No frizzing or curling was found up to 2 inches from the root of the hairs. Scatter and wind free-scatter response was tested to determine the silkiness and smoothness of the hair. The hair flow responded just like natural straight silky hair does.

In a preferred application, the hair is wetted and washed, then towel dried, and then the composition is applied directly onto the hair but not on the scalp, preferably about a one quarter inch from the scalp. Strands of hair are collectively coated with the conditioner and run evenly with a comb. After blow drying, the hair is then straightened with the thermal comb, and it then becomes silky, straight and smooth to the touch without the need for hair dryer application, or thermal straightening. For a period of three to six months, the hair remains straight and silky even after repeated washing without the need for re-straightening or using a blow drier to straighten hair.

The instant invention has been shown and described herein in what is considered to be the most practical and preferred embodiment. It is recognized, however, that departures may be made therefrom within the scope of the invention and that obvious modifications will occur to a person skilled in the art.

What I claim is:

1. A hair straightening and conditioner composition consisting essentially of:
(a) a leave-in conditioner;
(b) bio-extenders;
(c) marine algae;
(d) vitamin E;
(e) silicone;
(f) orange extract;
(g) formaldehyde;
(h) formic acid;
(i) citric acid;
(j) water;
(k) alcohol; and
(l) stearalkonium chloride.

2. A composition according to claim 1, further including an anti-bacterial solution.

3. A composition according to claim 1, further including a fragrance.

4. A composition according to claim 1, further including human keratin.

5. A treatment method for straightening and conditioning hair comprising the steps of:
(a) washing the hair;
(b) towel drying the hair;
(c) applying a composition to the hair and combing the hair to provide an even application, said composition including: a leave-in conditioner; bio-extenders; marine algae; vitamin E; silicone; orange extract; formaldehyde; formic acid; citric acid; water; alcohol; and stearalkonium chloride;
(d) blow drying the hair; and
(e) straightening the hair using a thermally heated comb.

6. A treatment method for straightening and conditioning hair according to claim 5, wherein said composition is applied approximately ¼″ from the scalp.

7. A treatment method for straightening and conditioning hair according to claim 5, further including the step of:
(f) applying said composition to the straightened hair.
